(19) United States
(12) Patent Application Publication
(10) Pub. No.: US 2019/0005397 A1
(43) Pub. Date: Jan. 3, 2019

(54) ENERGY-EFFICIENT, MODULARIZED UNCERTAINTY QUANTIFICATION AND OUTCOME PREDICTION IN MOBILE DEVICES

(71) Applicant: THE REGENTS OF THE UNIVERSITY OF CALIFORNIA, Oakland, CA (US)

(72) Inventors: Todd Prentice COLEMAN, La Jolla, CA (US); Marcela MENDOZA, San Diego, CA (US)

(21) Appl. No.: 16/063,228

(22) PCT Filed: Dec. 16, 2016
(86) PCT No.: PCT/US2016/067318

§ 371 Date: Jun. 15, 2018

Related U.S. Application Data
(60) Provisional application No. 62/268,427, filed on Dec. 16, 2015.

(51) Int. Cl. G06N 5/04; G06N 7/00; G16H 20/13; G06F 17/18; A61B 5/0432; A61B 5/00

(52) U.S. Cl. CPC G06N 5/045; G06N 7/005; A61B 5/0006; G06F 17/18; A61B 5/0432; G16H 20/13

(57) ABSTRACT

Energy-efficient, modularized systems and method for local uncertainty quantification and outcome prediction in mobile devices are disclosed. For example, an exemplary system implementing the disclosed technology includes a mobile device with at least one sensor and a processor sitting within an energy-efficient architecture. The processor runs an uncertainty quantification (e.g. Bayesian inference) algorithm on the data collected by the sensor and characterizes the uncertainty (e.g. the full posterior distribution) around latent variables of interest. The architecture for this algorithm is de-centralized, involves simple energy-efficient procedures that are implemented in parallel and in an iterative fashion, that allows for an aggregately fast, precise, and energy-efficient hardware embodiment of uncertainty quantification. Full quantification of uncertainty in estimates enables more robust predictions and decision-making. A statistically complete representation of the data can then be sent to a human, to an actuator, or to a cloud server for subsequent decision making.

### CROSS-REFERENCE TO RELATED APPLICATIONS

This patent document claims benefit of priority of U.S. Provisional Patent Application No. 62/268,427 entitled “ENERGY-EFFICIENT, MODULARIZED UNCERTAINTY QUANTIFICATION AND OUTCOME PREDICTION IN MOBILE DEVICES,” filed on Dec. 16, 2015. The entire content of the aforementioned patent application is incorporated by reference as part of the disclosure of this patent document.

### TECHNICAL FIELD

This patent document relates to uncertainty quantification.

### BACKGROUND

Currently, many mobile systems that acquire data from sensors rely upon transmission (e.g., wirelessly) of acquired data to external devices with high computational capabilities that perform uncertainty quantification and decision-making. For example, many mobile devices simply acquire data, digitize the data, and wirelessly transmit the data to a cloud server. However, wireless transmission of data is one of the most energy consuming tasks in these mobile devices. Moreover, in many mobile devices, the output of any uncertainty quantification and/or decision-making is given back to the human user or the actuator at the mobile device. Also, latency incurred in wireless transmission can be problematic for time-critical applications where the timeliness of a decision based upon the quantified uncertainty is crucial.

However, computing a posterior distribution is not always obtainable. Methods such as Markov Chain Monte Carlo and Variational Bayes have been used to approximate posterior distributions and provide uncertainty quantification of the decisions performed with the data but these methods may not be scalable or readily amenable to use in mobile applications.

### SUMMARY

The exemplary embodiments overcome the drawbacks of previous technologies by providing a method to compute a statistically complete representation of the uncertainty in desired unobserved parameters (e.g., the posterior distribution) locally and in an energy-efficient manner. The exemplary embodiments send only (if any) the most pertinent information to, for example, a human, an actuator, or a cloud server. Within a Bayesian context, the complete representation of the statistical uncertainty in the unobserved parameters can be utilized to attain an optimal decision (e.g. one that minimizes an expected cost). In some contexts, the representation of the uncertainty can be transmitted and an optimal decision can be computed remotely. In other contexts, aspects of the uncertainty can be provided to a human being so that they make a decision. In yet other contexts, the method for performing optimal decision making can also be implemented on the mobile device, and thus a closed loop system of measurement, inference, and decision making can be implemented with minimal (if any) need of transferring data for remote calculations.

In an exemplary embodiment, a mobile device is disclosed for determining uncertainty quantification of biometric data, the mobile device comprising: one or more sensors capable of collecting biometric data, a processing unit electrically coupled to the one or more sensors and capable of executing an uncertainty quantification algorithm on the biometric data collected by the one or more sensors, a wireless transceiver electrically coupled to the processing unit, and a display operatively connected to the processing unit.

In an exemplary embodiment, the uncertainty quantification algorithm is capable of finding a posterior distribution.

In an exemplary embodiment, the wireless transceiver is capable of wirelessly sending a representation of the posterior distribution to a cloud server.

In an exemplary embodiment, an actuator can be capable of one or more of receiving a representation of the posterior distribution, performing an action, or outputting a signal received by the mobile device. In an exemplary embodiment, the actuator capable of performing the action can include calculating an optimal action based upon the posterior distribution.

In an exemplary embodiment, the actuator includes any one or more of a speaker, an visual display, a drug delivery mechanism, and an electrical stimulator.

In an exemplary embodiment, the display is enabled to represent the posterior distribution for interpretation.

In an exemplary embodiment, the uncertainty quantification algorithm includes a Bayesian inference algorithm.

In an exemplary embodiment, the one or more sensors comprise electrocardiograph (EKG) monitors.

In an exemplary embodiment, the one or more sensors comprise adhesive-integrated flexible electronics for recording physiologic signals.

In an exemplary embodiment, the one or more sensors comprise electroencephalograph (EEG) epidermal electronics.

In an exemplary embodiment, the one or more sensors are enabled to measure one or more of maternal temperature, fetal heart rate, fetal movement, or uterine contractions.

In an exemplary embodiment, the uncertainty quantification algorithm is capable of determining a quantification of uncertainty.

In an exemplary embodiment, one or more of the display or the sensors are enabled to display an alert based on the quantification of uncertainty. For example, one or more of the display or the sensors are enabled to display an alert based upon decision-making method that takes as input the quantification of uncertainty

In an exemplary embodiment, the one or more of the display or the sensors is enabled to display a green light when the quantification of uncertainty is within a range. For example, the green light can be displayed when a function of the quantified uncertainty is within a range.

In an exemplary embodiment, the one or more of the display or the sensors is enabled to display a yellow light when a function of the quantified uncertainty is close to a threshold of a range.

In an exemplary embodiment, the one or more of the display or the sensors is enabled to display a red light when a function of the quantification of uncertainty is outside a range. For example, the red light can be displayed when a function of the quantified uncertainty is outside a range.

In an exemplary embodiment, the processing unit is enabled to receive and process one or more of tolerance settings or a range of a quantification of uncertainty from a remote device.

In an exemplary embodiment, the processing unit includes a plurality of modules wherein a first set of one or more modules are enabled to in parallel implement point estimation, and a second set of one or more modules are operatively connected to an output of the first set of one or more modules, wherein the second set of one or more modules are enabled to aggregate results of the first set of one of more modules and provide the aggregated results to the first set of one or more modules. In an exemplary embodiment, the point estimation comprises solving LASSO problems.

In an exemplary embodiment, the plurality of modules includes one or more analog solvers.

In an exemplary embodiment, the processing unit includes a graphic processing unit.

In an exemplary embodiment, the processing unit includes a processor.

In an exemplary embodiment, the processing unit is enabled to execute one or more of voice commands or voice recognition.

In an exemplary embodiment, the biometric data includes physiologic time series data.

In an exemplary embodiment, a method for determining uncertainty quantification of biometric data implemented by the exemplary mobile device.

In an exemplary embodiment, a computer-readable program storage medium having code stored thereupon, the code, when executed by a processor, causing the processor to implement the method recited for the exemplary mobile device.

In an exemplary embodiment, it is desirable to reduce wireless transmission of data while conserving a robust manner of representing the data at least because the performance of the mobile device is constrained by its battery energy.

In another exemplary embodiment, a huge savings in terms of latency, energy, privacy, and security is advanced if there is an opportunity to bypass the need to wirelessly transmit to an external device, process data, and then have the results transmitted from the external device back to the mobile device.

Disclosed are energy-efficient, modularized systems and methods for local uncertainty quantification and outcome prediction in mobile devices.

In an exemplary embodiment, the disclosed technology for local uncertainty quantification and outcome prediction can be implemented in a system with one or more sensors with energy limitations and wireless connectivity to a mobile device, the cloud, or both.

In another exemplary embodiment, the disclosed technology for local uncertainty quantification and outcome prediction can be implemented in a mobile device with (less stringent as compared to the system with one or more sensors) energy limitations and wireless connectivity to the cloud.

In yet another exemplary embodiment, the disclosed technology for local uncertainty quantification and outcome prediction can be implemented in the cloud with virtually unlimited processing capability.

In general, a system with one or more sensors and mobile devices tend to perform limited processing. The disclosed technology enables in such sensors and mobile devices more sophisticated data interpretation methods that quantify uncertainty and either directly alert the user at the sensors, with wireless transmission to the mobile devices, or wireless transmission to the cloud only when the uncertainty is such that a human needs to help with interpretation of the situation or it has been determined there is an emergency. In an exemplary embodiment, mobile devices and sensors can analyze data in a local manner and perform uncertainty quantification on the estimable parameters of interest. This in turn can enable the device to give feedback to a human user at specific times about the interpretability of the parameters. For example, the device can give feedback to a human user about physiologic time series data (e.g. heart rate, temperature, brain rhythms, pregnancy monitoring, etc.) collected with wearable sensors. In addition, the disclosed system enables for alerting a user or a physician whenever there is an emergency as determined by the analysis of the data.

The disclosed technology can be used to quantify uncertainty at mobile devices without wireless transmission to the cloud only when the uncertainty is such that a human needs to help with interpretation of the situation or it has been determined there is an emergency.

The disclosed technology can allow sensors and mobile devices to perform processing of a large class of uncertainty quantification and approaches that deal with physiologic time series data (e.g. heart rate, temperature, brain rhythms, pregnancy monitoring, etc.) that can be measured with small wearable sensors, which are not typically processed by the device.

In an exemplary embodiment, the disclosed system can include an actuator that is activated by the device when an event occurs. For example, an insulin administration can be given to a diabetic patient whenever the device detects that glucoses levels have exceeded a predefined threshold. In general, a device can perform optimal decision making as it achieves an accurate representation of the data by computing, for example, a posterior distribution.

In an exemplary embodiment, the disclosed technology can be implemented using architectures based on digital or analog circuits. These architectures perform mathematically precise computation of desired uncertainty parameters, and in a manner that is computationally efficient. Specifically, the exemplary embodiment comprises many parallel sub-systems that implement methods for point estimation, and then aggregate. Each individual sub-system in parallel can for example comprise one of many existing energy-efficient and low-latency methods for point estimation. The disclosed technology utilizes any such existing method for point estimation, integrates many such methods on parallel systems that pass messages iteratively with efficient linear algebra aggregation steps so that after multiple iterations, a precise quantification of uncertainty (e.g. posterior distribution) is represented as a set of coefficients of polynomials. These coefficients can then be used to draw statistically independent samples from a posterior distribution. In the exemplary embodiment, the independent samples from the posterior can be combined with a cost function to identify a decision that minimizes expected cost. In other settings, a point estimate can be appended with “error bars” or its “uncertainty profile”. When this uncertainty profile is within a specified range, this can be deemed “normal” and a “green light” is provided on the sensor to the user. However, if the point estimate along with it “uncertainty profile” lie outside a pre-specified range, then this is deemed abnormal and an feedback indication, such as a “red light” is provided to the user. Similarly, there can be an intermediate other indicators, such as a “yellow light”. In yellow and red scenarios, the sensor then and only then can transmit wirelessly to a smart phone or to the cloud. Because this happens much less often, much more energy savings can be achieved.

Moreover, the aforementioned exemplar embodiment allows for a red light to be immediately given to a human user so that they may take appropriate action. This can provide great utility, for example if someone wearing an EKG monitors is at risk of a heart malfunction; the red light indication can alert the user in real to get medical attention. The same analogy holds for pregnancy monitoring; a pregnant woman with a high risk pregnancy can wear a mobile sensor that notifies here when the fetal heart rates and uterine contractions give rise to an emergency where an obstetrician should be contacted. In this setting, the full uncertainty quantification can identify more information about the status of the pregnancy beyond simply a point estimate: a function of uncertainty and its relationship to a threshold can help determine of contacting an obstetrician is necessary. Moreover, the decision to alert a pregnant woman or not, when implemented with a Bayes optimal decision making strategy that averages across samples taken from the posterior distribution, will result in lower false positive rates for the same level of false negatives. If the same level of false positive and false negative rates were desired with current existing approaches, the streaming of all the data to a non-worn mobile device would result in a huge battery requirement—thus questioning notion of a “wearable” because of its requirement for a large battry. The disclosed technology providing an extensible framework to quantify uncertainty and affort optimal decision making, without the need for persistent transmission to a non-worn mobile device, thus resulting in less energy requirements, smaller batteries, and smaller architectures that are more likely to be adopted as truly “wearable.”

The disclosed technology can apply to a large class of physiologic processes—not specifically one. The algorithm and the underlying sub-algorithm of each parallel unit can be configured in a context-specific manner to perform appropriate estimation and uncertainty quantification.

In addition, the exemplary technique according to the disclosed technology can enable a remote device (e.g. from a physician's console, or from the internet or from another mobile device) to adjust parameters pertaining to computation of the optimal decision making method. For example a remote device can reconfiguring the thresholds for when functions of the uncertainty render one decision or another (e.g. red, green, yellow). The disclosed technology can enable bi-directional communication between the sensors, mobiles devices, and the cloud to allow such reconfiguration.

In another aspect, the disclosed technology can be used for auditory processing applications. For example, many current voice recognition and command systems with intelligent systems on mobile phones or tablets acquire data voice data from the user, then send it to the cloud for interpretation the data, and then send it back to the mobile device. This latency can adversely affect the human experience. The disclosed technology can allow for optimal Bayesian classification by first computing the uncertainty in latent parameters, and then drawing independent samples from their posterior distribution to minimize an expected loss function pertaining to optimal classification. Using the disclosed methods, the voice recognition and command system in the mobile phone/tablet will no longer be adversely affected by the need to transfer data back and forth from the cloud, and thus lead to improved user experience, while still affording optimal Bayesian classification performance. In the event of a medical context, this saving in latency can be the difference between life and death.

The disclosed technology can be implemented to provide a method of coalescing N chips, cores, or modules that perform “simple,” “dumb” estimation. By carefully interconnecting and passing messages back and forth between the N chips, cores, or modules, the disclosed technology can achieve a sophisticated, “smart” estimation aggregate system. The disclosed technology can not only estimate an underlying signal from a sensor's noisy measurements, but it also can quantify its uncertainty.

The disclosed technology can allow the aggregate size and energy expenditure of the aggregate system to be small and energy efficient. For example, the disclosed technology can be implemented on analog integrated circuit architectures that are extremely small spatially as well as in terms of energy expenditure.

The disclosed technology can enable embedding these small architectures unobtrusive wearable devices with low energy expenditure.

The disclosed technology can enable a framework of interpretation of data, with uncertainty profiles, and stratification in terms of alerts, such as red, yellow, and green lights.

The disclosed technology can enable a framework of an adaptive way to intermittently, only when statistically necessary, move data back and forth for more high-power computational, human interpretation, or both.

The disclosed technology enables a framework for a human or cloud to adjust the tolerance settings remotely, based upon data that has been collected so far.

Exemplary systems implementing the disclosed technology can be applied to a large class of uncertainty quantification and approaches that deal with physiologic time series data (e.g. heart rate, temperature, brain rhythms, pregnancy monitoring, etc.). For example, the disclosed technology can be applied to multiple EEG physiologic signals (sleep and attention features).

### BRIEF DESCRIPTION OF THE DRAWINGS

FIG. 1A shows a conventional wireless transmission schemes where signals are acquired and wirelessly transmitted.

FIG. 1B shows an exemplary analog-to-information scheme where inference is performed locally and only the posterior distribution is transmitted.

FIG. 1C shows an exemplary schematic of proposed system and method for uncertainty quantification prediction, uncertainty outcome prediction, or both.

FIG. 2 shows an exemplary schematic of a processor in a mobile device.

FIG. 3 shows calculating the posterior distribution with a plurality of analog solvers.

FIG. 4 shows an exemplary sensor and an exemplary detection of alpha waves.

FIG. 5A shows exemplary posterior samples over three of seven frequency bands generated from EEG windows during REM and light sleep.

FIG. 5B shows an exemplary histogram of losses of Bayesian LASSO (Least Absolute Shrinkage and Selection Operator) vs. LASSO decisions.

FIG. 6 illustrates a comparison of linear regression estimates on diabetes data LASSO, MCMC, and the exemplary method trace plots for estimates of the diabetes data regression parameters.

FIG. 7 illustrates an exemplary block diagram of the various components of exemplary device.

FIG. 8 illustrates an exemplary flow diagram of the exemplary device.

### DETAILED DESCRIPTION

I. Introduction

As high-dimensional and complex datasets become the norm, uncertainty quantification is crucial to decision-making applications. It is imperative to “have error bars in all our predictions,” as statistician Michael Jordan has expressed. From a Bayesian point of view, an accurate way to represent uncertainty and minimize risk in decision-making is via the posterior distribution. However, a way of accurately calculating the posterior has been traditionally unobtainable.

Bayesian inference can be cast as a problem of finding a nonlinear map that transforms samples from the prior to samples from the posterior. Under log-concavity assumptions, a Kullback-Leibler (KL) divergence minimization framework results in a convex optimization problem. The latter problem can be iteratively solved using a series of quadratically-regularized convex point estimation problems.

The exemplary embodiments can be implemented for applications in which a latent signal can be modeled as sparse. This is a natural model for many applications in statistics, signal processing, and compressed sensing. In practicality, these applications solve a sparse approximation, or LASSO, problem which reconstructs vectors in terms of a basis to obtain a sparse representation of the input. Many efficient algorithms for solving LASSO have been proposed over the years. Moreover, recent work has introduced a class of analog-implementable LASSO solvers that open the path to energy-efficient computations in hardware. However, LASSO solutions are point estimates and thus lack the ability to quantify the uncertainty associated with their approximations. Work in the past has introduced Bayesian LASSO, a way to calculate the posterior which relied on Markov Chain Monte Carlo methods, but these methods remain non-scalable and thus limit their implementation in many applications.

Emerging applications such as wearable electronics and the internet-of-things not only generate large and high-dimensional data but necessitate wireless transmission of these datasets. As an example, consider an ultrasound-on-a-chip idea in which numerous sensors at the acquisition end generate large and high-dimensional data. The standard protocol is to digitize and wirelessly transmit sensor signals to an external server for analysis and processing. Even under substantial compression of data, this communication flow leads to high energy costs and exposes devices to security attacks. It is therefore desirable to bypass large-scale transmission of data and transmit a concise and complete representation of the data (such as the posterior) only at infrequent events.

This patent document discloses, among other features, that:
1) Bayesian LASSO can be solved by linear algebra updates and a series of LASSO problems. Further, a quadratically regularized point estimation problem is equivalent to LASSO.
2) The framework is instantiated with a low-energy, analog-implementable solver with results shown.

With this framework an ‘analog-to-information’ framework can be implemented in which the posterior is calculated locally within a device and only a few variables representing the posterior are wirelessly transmitted in the event of abnormality, obviating the need to transmit large data sets.

Emerging applications such as wearable electronics and the internet-of-things necessitate energy-efficient frameworks for processing large and high-dimensional data. The standard protocol is to digitize and wirelessly transmit sensor signals to an external server for analysis of these datasets. Even under substantial compression of data, this communication flow leads to high energy costs. It is therefore desirable to bypass large-scale transmission of data and transmit a concise and complete representation of the data only at infrequent events.

A framework is considered for a complete representation for sparse representation of signals, a standard tool in signal processing. The most widely used algorithm in sparse approximation is the LASSO (Least Absolute Shrinkage and Selection Operator) which simultaneously induces shrinkage and sparsity in the estimation of regression coefficients. The formulation of the standard LASSO is as follows:

$${x}^{*} = {arg min}_{x ∈ {ℝ}^{d}} {‖ y - Φ x ‖}_{2}^{2} + λ {‖ x ‖}_{1} \qquad ( 1 a )$$

where y ∈ [symbol] ⁿ is a vector of responses, Φ is a n×d matrix of standardized regressors, and x ∈ [symbol] ^{d} is the vector of regressor coefficients to be estimated.

A variety of algorithms for solving the standard LASSO problem are typically applied including iterative soft-thresholding and its successors. It has been observed that the LASSO can be interpreted as a Bayesian posterior mode estimate with a particular prior. However, obtaining a point estimate of a posterior distribution such as the mode cannot provide information about the uncertainty of the estimates. A fully Bayesian approach can not only provide methods for finding point estimates but also leads to optimal decision making, risk minimization, and uncertainty quantification of the lasso regression coefficients via confidence intervals. Transmitting the parameters that specify a posterior distribution can lead to a concise and statistically complete representation of the data therefore reducing transmission overheads.

However, computing the Bayesian LASSO posterior is not generally tractable.

Furthermore, this patent document addresses the need for scalability and energy-efficiency by introducing a parallelizable Bayesian Lasso that can be implemented in energy-efficient architectures. Producing a posterior distribution has been traditionally expensive both in a computational and energy sense. Markov Chain Monte Carlo methods are sequential in nature, thus often do not scale well with dataset size or model complexity. In contrast, a scalable and parallel framework for Bayesian inference can be utilized using a measure transport methodology. In an exemplary embodiment, these methods are adapted to the Bayesian Lasso.

Finally, in an exemplary embodiment, Bayesian Lasso can be implemented in computationally and energy-efficient architectures. Graphics Processing Cards have been recently used to alleviate the scalability issues in MCMC algorithms and accelerate Gibbs sampling methods. However, widespread adoption of GPU accelerated sampling remains a challenge, as these algorithms require high level functions that are not provided by the current low-level nature of GPU programming languages.

In the exemplary embodiments, the proposed Bayesian Lasso can be implemented in a GPU and an energy-efficient analog-solver.

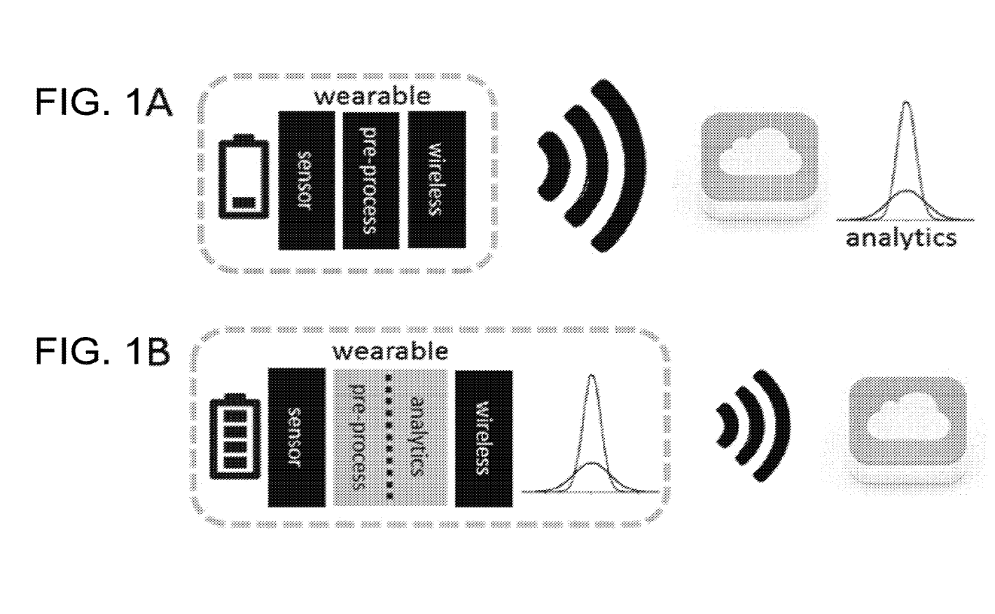

FIGS. 1A and 1B shows applications where the analytics are performed at the wearable end instead of in external servers. FIG. 1A shows exemplary wireless transmission schemes where signals are acquired and wirelessly transmitted. FIG. 1B shows an exemplary analog-to-information scheme where inference is performed locally and only the posterior distribution is transmitted.

Examples of implementations of the disclosed technology can provide for an energy-efficient, modularized system and method for local uncertainty quantification and outcome prediction in mobile devices. For example, as shown in FIG. 1C, an exemplary system (100) implementing the disclosed technology includes a mobile device (102) that can include at least one sensor (104) and a processor (106) sitting within an energy-efficient architecture. The processor can run an uncertainty quantification (e.g. Bayesian inference) algorithm on the data collected by the sensor and can characterize the uncertainty (e.g. the full posterior distribution (108)) around latent variables of interest. The architecture for this algorithm can be de-centralized, and can involve simple energy-efficient procedures that are implemented in parallel and in an iterative fashion, so that it can allow for an aggregately fast, precise, and energy-efficient hardware embodiment of uncertainty quantification. Full quantification of uncertainty in estimates enables more robust predictions and decision-making. As shown in FIG. 1C, a statistically complete representation of the data (e.g. a succinct parameter that enables sampling from the posterior distribution) can then be sent to a human (114), to an actuator (112), or to a cloud server (116) for subsequent decision making.

FIG. 1C illustrates an exemplary schematic of proposed system and method for uncertainty quantification/outcome prediction. FIG. 1C illustrates a mobile device (102) with a sensor (104) to collect data to compute a posterior distribution using, for example, a processor (106) in an energy efficient manner. Using the posterior distribution, the processor (106) can send a summarized report of the state of the system to an actuator (112), to a human (114), or to a cloud server (116) by using, for example, a wireless transmitter (110). In the case of transmission to the actuator (112), the actuator (112) can perform an action and its output is sensed by the device in a feedback loop.

The exemplary method is modularized and is universally applicable to different types of sensing, different statistical models, and is thus applicable to many different contexts (eg. Speech, video, physiologic monitoring, etc.). This modularization enables encompassing different statistical models within a single energy-efficient architecture.

The exemplary methods reduce the reliance of the mobile device on wireless communication, which is very energy costly, to allow for external systems to perform uncertainty quantification. In an exemplary embodiment, when a mobile system includes an exemplary embedded architecture, an actuator (e.g., a speaker, an LED, a drug delivery mechanism, an electrical stimulator, etc.), such an exemplary system can allow for a closed-loop sensing, interpretation, and actuation without the need for relaying information wirelessly to an external device. Such an exemplary system can provide energy savings as well as security advantages.

Computing the full posterior distribution enables uncertainty quantification and robust outcome prediction. However, obtaining the full posterior distribution in a Bayesian sense is traditionally difficult, since leading algorithms to compute the posterior are computationally inefficient and/or non-scalable. For example, classification algorithms developed in hardware traditionally rely on computing only the most likely explanation of the data and do not quantify uncertainty because of the computational costs to do so with existing architectures. Such algorithms are more error-prone because estimates do not quantify uncertainty for robust classification. Other hardware architectures wirelessly transmit acquired data to the cloud and allow for sophisticated algorithms to be implemented there, but such systems incur latency, privacy and energy short-comings.

In an exemplary embodiment, a method is introduced to compute the full posterior distribution in a parallelizable manner that can be implemented in energy-efficient architectures. This modularized method is precise, energy-efficient, and obviates the need for wireless transmission. Moreover, the exemplary modularized approach applies to many statistical models and contexts, thus possessing the ability to solve a variety of statistical models with a single energy-efficient architecture. This differs from the leading algorithm to compute the posterior: Markov Chain Monte Carlo, as changing the statistical model changes the formulation of the algorithm drastically.

This exemplary framework can reside within a mobile device that collects data from a subject or from the environment. The state of the subject or environment can be inferred in a robust manner within the processor of the device and only a representation of the posterior distribution is transmitted when necessary.

Furthermore, the exemplary embodiment can include at least one sensor for collection of data of interest. The sensor(s) communicates with a processor directly or via a preprocessor. The processor can run an optimization algorithm to calculate the posterior of the data from the sensor.

II. The Processing Unit

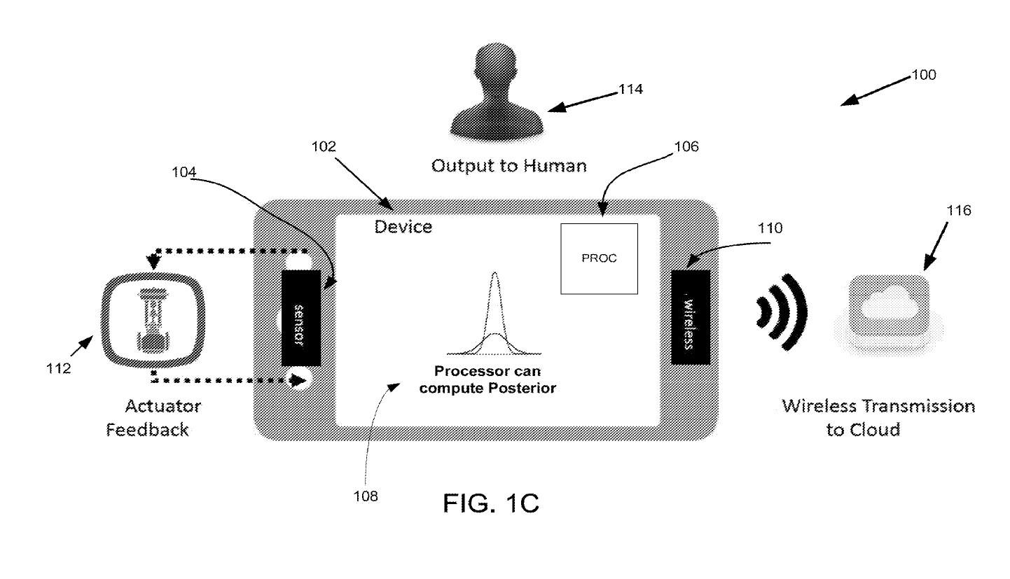

FIG. 2 illustrates an exemplary schematic of a processor in a mobile device. As illustrated in an exemplary embodiment of FIG. 2, the optimization to compute the posterior is decomposed into modules that can run in parallel. The processor (204) is comprised of N modules. The N-1 modules (208) can be identical, can have the exact same function, and can run in parallel. The Nth module (210) is context specific and aggregates the N-1 modules' results. This flow proceeds in an iterative fashion until the optimization solution converges.

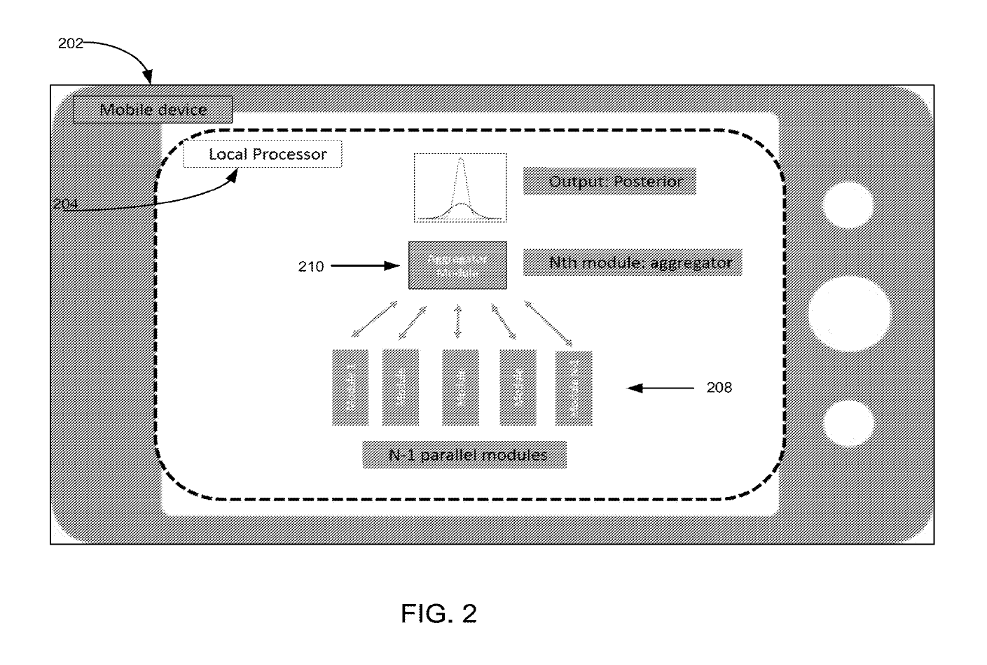

FIG. 3 illustrates an exemplary embodiment for calculating the posterior distribution with the exemplary analog solvers. The exemplary processor (302) with N components can have N-1 components (304) comprised of equivalent circuit analog solvers. As illustrated in an exemplary embodiment of FIG. 3, the N-1 modules (304) can be comprised of analog circuits (306) that work in parallel, lie within a parallelized Graphics Processing Unit solution, or make the use of another energy-efficient architecture. The first N-1 modules can solve a single part of the optimization and send their solutions to the Nth module (the aggregator module) (308). This flow can continue in an iterative manner until the Bayesian algorithm has converged. Then the processor (302) can output a representation of the posterior distribution to be sent to the actuator, a human, or to a cloud server as shown in the exemplary embodiment of FIG. 1C.

III. Bayesian Lasso in a Distributed Architecture

The exemplary embodiments disclosed herein include a distributed framework for finding the full posterior distribution associated with LASSO (Least Absolute Shrinkage and Selection Operator) problems. The exemplary embodiments can leverage the results of formulating Bayesian inference as a Kullback-Leibler (KL) divergence minimization problem that can be solved with linear algebra updates and a series of convex point estimation problems. The exemplary embodiments show that drawing samples from the Bayesian LASSO posterior can be done by iteratively solving LASSO problems in parallel. Motivated by wearable applications where (a) the energy cost of continuous wireless transmission is prohibitive and (b) cloud storage of data induces privacy vulnerabilities, the exemplary embodiments include a class of ‘analog-to-information’ architectures that only transmit the minimal relevant information (e.g., the posterior) for optimal decision-making. This result can be instantiated with an analog-implementable solver and the posterior can be calculated with systems of low-energy analog circuits in a distributed manner.

The Bayesian LASSO renders the posterior distribution for the Lasso problem and is traditionally computed via Gibbs sampling. However, Gibbs sampling methods suffer from lack of scalability and samples from this methodology are necessarily correlated. An exemplary measure transport approach is provided to compute uncorrelated samples from the Bayesian Lasso posterior that is distributed and only requires a series of Lasso solvers and linear algebra solvers. Inspired by applications in wearable electronics, This formulation is amenable to implementation in computing systems that leverage parallelization and architectures that are energy-efficient.

IV. Lasso and Bayesian Lasso

The following generative model show how a latent and sparse X ∈ [symbol] ^{d} relates to a measurement Y ∈ [symbol] ⁿ:

Y=ΦX+∈ (1b)

and the measurement noise satisfies ∈ ˜ [symbol] (0, σ²I). An i.i.d. Laplacian statistical model X is assumed; specifically, for any X ∈ [symbol] ^{d}:

$$p ( x ) = {\prod }_{i = 1}^{d} \frac{α}{2} {e}^{- α \vert {x}_{i} \vert} , x ∈ {ℝ}^{d} \qquad ( 2 )$$

Throughout this discussion, it is assumed that Φ and α are fixed and known. The negative log posterior density of X given Y=y satisfies

−log p(x|y)×∥y−Φx∥₂²+λ∥x∥₁ (3)

where λ is a ratio between α and σ². As such, the standard LASSO problem

$${x}^{*} = {arg min}_{x ∈ {ℝ}^{d}} {‖ y - Φ x ‖}_{2}^{2} + λ {‖ x ‖}_{1} \qquad ( 4 )$$

is a maximum a posteriori estimation problem for the Laplacian prior in (2).

Imposing a Laplacian prior is equivalent to L₁-regularization, which has desirable properties, including robustness and logarithmic sample complexity. Various algorithms for solving (4) are typically applied including iterative soft-thresholding and its successors. These methods are scalable, yet only provide point estimates. For optimal Bayesian decision making, the full posterior distribution (or a way to draw i.i.d. samples from the posterior) is required. A framework is considered to be able to generate samples Z₁, . . . , Z_{K} from the posterior distribution on X, given by (3). With this information, for any set of possible decisions [symbol] , and any loss function T: [symbol] ^{d}× [symbol] → [symbol] , the Bayes optimal decision d* (y) can be performed by minimizing the (appropriate) conditional expectation, given by:

$${arg min}_{d ∈ 𝒟} 𝔼 [ l ( X , d ) \vert Y = y ] ≃ {arg min}_{d ∈ 𝒟} \frac{1}{N} {\sum }_{k = 1}^{K} l ( {z}_{k} , d ) \qquad ( 5 )$$

Other approaches have been developed involving Markov Chain Monte Carlo algorithms, however the samples Z₁, . . . , Z_{K} are necessarily correlated. Here, a scalable way to generate Z₁, . . . , Z_{K} is presented so that they are i.i.d. from the posterior, are exact, and are built upon a convex optimization formulation that can be solved in a distributed manner.

The Bayesian inference is casted as a problem of finding a diffeomorphism S: [symbol] ^{d}→ [symbol] ^{d} that pushes the prior in (2) to the posterior of the Bayesian Lasso.

Definition II.1. Define the density of the prior as p and the density of the posterior as q. A map S pushes p to q, i.e. it transforms a sample W from p into a sample Z−S(W) from q.

V. Distributed Bayesian Sparse Approximation

This section provides some background and solves Bayesian LASSO by solving, in parallel, a batch of LASSO problems, which themselves can be solved with existing sparse approximation algorithms. This section also provides background on measure transport theory and show that a map S can be found that pushes samples from the prior to the Bayesian Lasso posterior. The ADMM framework can be utilized to develop a distributed Bayesian LASSO solver. Furthermore, the Bayesian LASSO can be formulated as a batch of LASSO problems, which themselves can be solved with existing sparse approximation algorithms in a parallel manner.

A. Convex Optimization and Fully Bayesian Inference

For the problems where the prior and likelihood are log-concave, developing a map that transforms i.i.d. samples from the prior into i.i.d. samples from the posterior can be performed with convex optimization. Here, that background information is provided.

The starting point is that all monotonic diffeomorphisms are considered as the set of maps with positive-definite Jacobian:

[symbol]

+ [symbol] {S: [symbol] ^{d}→ [symbol] , J_{S}>0}

If density of the prior is defined asp and the density of the posterior is defined as q, then it is known that there always exists an [symbol] + that pushes p to q, e.g., it transforms a sample W from p into a sample Z=S* (W) from q. Given an arbitrary [symbol] +, the pₛ for which S pushes \tilde{p}_{S} to q is given by the Jacobian equation:

\tilde{p}_{S}(u)=q(S(u)) det (J_{S}(u)) for all u ∈ [symbol] ^{d} (6)

By defining

g(z) [symbol] −[log f_{Y|X}(y|z)+log f_{X} (z)] (7)

S* satisfies the following Kullback-Leibler (KL) divergence variational equation:

$$\begin{matrix}{S}^{*} = {arg min}_{S ∈ {ℳ𝒟}_{+}} D ( P \vert \vert {\tilde{P}}_{S} ) \\ = {arg max}_{S ∈ {ℳ𝒟}_{+}} {𝔼}_{P} [ - g ( S ( X ) ) + log det ( {J}_{S ( X )} ) ]\end{matrix} \qquad \begin{matrix}( 8 ) \\ \\ ( 9 )\end{matrix}$$

Moreover, when q is log-concave (equivalently when g is convex), this (infinite-dimensional) optimization problem is convex.

Polynomial Chaos Expansion: Any [symbol] + is approximated as a linear combination of basis functions through a Polynomial Chaos Expansion (PCE), where ∅ are the polynomials orthogonal with respect to the priorp:

$$\begin{matrix}S ( x ) = {\sum }_{j ∈ 𝒥} {b}_{j} {φ}^{( j )} ( x ) & ( 10 ) \\ {\int }_{x ∈ X} {φ}^{( i )} ( x ) {φ}^{( j )} ( x ) p ( x ) dx = {δ}_{i , j} & ( 11 )\end{matrix}$$

with δ_{i,J} being 1 if i=j and 0 otherwise. Now define K= [symbol] and we have that for X ⊂ [symbol] :

$$\begin{matrix}\begin{matrix}F = [ {b}_{1} , … , {b}_{K} ] , & d \times K\end{matrix} & ( 12 ) \\ \begin{matrix}A ( x ) = {[ {φ}^{( 1 )} ( x ) , … , {φ}^{( K )} ( x ) ]}^{T} , & K \times 1\end{matrix} & ( 13 ) \\ \begin{matrix}S ( x ) = FA ( x ) , & d \times 1\end{matrix} & ( 14 ) \\ \begin{matrix}J ( x ) = {[ \frac{\partial {φ}^{( i )}}{\partial {x}_{j}} ( x ) ]}_{i , j} , & K \times d\end{matrix} & ( 15 ) \\ \begin{matrix}{J}_{S} ( x ) = FJ ( x ) & d \times d .\end{matrix} & ( 16 )\end{matrix}$$

Then the expectation from (9) can be approximated using an empirical expectation based upon i.i.d. samples from p. Letting Aᵢ [symbol] A(Xᵢ) and Jᵢ [symbol] J(Xᵢ), we arrive at the following finite-dimensional problem:

$${F}^{*} = {arg max}_{F : {FJ}_{i} ≻ 0} \frac{1}{N} {\sum }_{i = 1}^{N} g ( {FA}_{i} ) + log det ( {FJ}_{i} ) \qquad ( 17 )$$

Whenever q is log-concave (equivalently g is convex), this is a finite-dimensional convex optimization problem. Moreover, as K→∝, from the PCE theory, the map F*A (x) converges to the optimal map S* that pushes p to q.

B. Parallelized Convex Solver With Admm

A scalable framework can be used to solve (17) which only requires iterative linear algebra updates and solving, in parallel, a number of quadratically regularized point estimation problems. The distributed architecture involves an augmented Lagrangian and a concensus Alternating Direction Method of Multipliers (ADMM) formulation:

$$\begin{matrix}{min}_{F , Z , p , B} & \frac{1}{N} {\sum }_{i = 1}^{N} g ( {p}_{i} ) - log det {Z}_{i} + & \frac{1}{2} ρ {‖ {F}_{i} - B ‖}_{2}^{2} \\ & + \frac{1}{N} {\sum }_{i = 1}^{N} \frac{1}{2} ρ {‖ {BA}_{i} - {p}_{i} ‖}_{2}^{2} + & \frac{1}{2} ρ {‖ {BJ}_{i} - {Z}_{i} ‖}_{2}^{2}\end{matrix}$$

$$\begin{matrix}s . t . & {BA}_{i} = {p}_{i} : & {γ}_{i} ( d \times 1 ) \\ & {BJ}_{i} = {Z}_{i} & {β}_{i} ( d \times d ) \\ & {F}_{i} - B = 0 : & {α}_{i} ( d \times K ) \\ & {Z}_{i} ≻ 0 & \end{matrix}$$

for any fixed ρ>0 .

A penalized Lagrangian is solved iteratively by first solving for B^{k+1}

$${B}^{k + 1} = \frac{1}{N} {\sum }_{i = 1}^{N} [ ρ ( {F}_{i}^{k} + {p}_{i}^{k} {A}_{i}^{T} + {Z}_{i}^{k} {J}_{i}^{T} ) + {γ}_{i}^{k} {A}_{i}^{T} + {β}_{i}^{k} {J}_{i}^{T} + {α}_{i}^{k} ] ℳ , \\ ℳ {=}^{Δ} {[ ρ ( I + \frac{1}{N} {\sum }_{i = 1}^{N} {A}_{i} {A}_{i}^{T} + {J}_{i} {J}_{i}^{T} ) ]}^{- 1} \qquad ( 18 )$$

and then solving, in parallel for 1≤i≤N, the other variable updates:

$$\begin{matrix} {F}_{i}^{k + 1} = - \frac{1}{ρ} {α}_{i}^{k} + {B}^{k + 1} & ( 19 a ) \\ {Z}_{i}^{k + 1} = Q {\tilde{Z}}_{i} {Q}^{T} & ( 19 b ) \\ {p}_{i}^{k + 1} = {arg min}_{{p}_{i}} g ( {p}_{i} ) + \frac{1}{2} ρ {‖ {B}^{k + 1} {A}_{i} - \vert {p}_{i} ‖}_{2}^{2} + {γ}_{i}^{kT} ( {p}_{i} - {B}^{k + 1} {A}_{i} ) & ( 19 c ) \\ {γ}_{i}^{k + 1} = {γ}_{i}^{k} + ρ ( {p}_{i}^{k + 1} - {B}^{k + 1} {A}_{i} ) & ( 19 d ) \\ {β}_{i}^{k + 1} = {β}_{i}^{k} + ρ ( {Z}_{i}^{k + 1} - {B}^{k + 1} {J}_{i} ) & ( 19 e ) \\ {α}_{i}^{k + 1} = {α}_{i}^{k} + ρ ( {F}_{i}^{k + 1} - {B}^{k + 1} ) & ( 19 f )\end{matrix}$$

ADMM guarantees convergence to the optimal solution. To emphasize, each ith update in (19) is parallel. As (19b) is an eigenvalue-eigenvector decomposition, it follows that all the updates involve linear algebra with the exception of (19c), which is a quadratically regularized point estimation problem.

C. Efficiently Solving the Bayesian Lasso

Here, the unique problem structure of Bayesian LASSO is exploited to simplify a scalable implementation.

1) Polynomial Chaos Expansion for Bayesian Lasso

Here, as shown, the prior distribution (Laplacian) for Bayesian LASSO has a closed-form PCE.

Lemma III.1. The PCE for the Laplacian distribution is ∅_{L} (x)=∅_{E} (|x|) where ∅_{E} are the Laguerre polynomials.

### Proof

$$\begin{matrix}{\int }_{- ∞}^{∞} {φ}_{E}^{i} ( \vert x \vert ) {φ}_{E}^{j} ( \vert x \vert ) {p}_{L} ( x ) dx = {\int }_{- ∞}^{∞} {φ}_{E}^{i} ( \vert x \vert ) {φ}_{E}^{j} ( \vert x \vert ) \frac{1}{2} {p}_{E} ( \vert x \vert ) dx \\ = 2 {\int }_{0}^{∞} {φ}_{E}^{i} ( x ) {φ}_{E}^{j} ( x ) \frac{1}{2} {p}_{E} ( \vert x \vert ) dx \\ = {δ}_{i , j}\end{matrix} \qquad ( 20 )$$

Where the first equality holds because the Laplacian density p_{L} (x) is related to the exponential density p_{E} (x) by

$${p}_{L} ( x ) = \frac{1}{2} {p}_{E} ( \vert x \vert ) ,$$

the second equality holds by symmetry of the function being integrated, and the third follows because the PCE for the exponential distribution is obtained with the Laguerre polynomials ∅_{E}⁽¹⁾.

2) Bayesian Lasso Via Admm

It is now shown that for Bayesian LASSO, the only ADMM update that is not linear algebra is simply a LASSO problem.

Theorem III.2. For the Bayesian LASSO statistical model given by (3), the ADMM update (19c) is a d-dimensional LASSO point estimation problem:

$${p}_{i}^{k + 1} = {arg min}_{{p}_{i}} {‖ \hat{y} - {\hat{Φ}}^{T} {p}_{i} ‖}_{2}^{2} + λ {‖ {p}_{i} ‖}_{1} \qquad ( 21 )$$

where \hat{Φ} and ŷ satisfy

$${\hat{Φ}}^{T} \hat{Φ} = {Φ}^{T} Φ + \frac{1}{2} ρ I \\ \hat{y} = {( [ {y}^{T} Φ + \frac{1}{2} {ρ ( {B}^{k + 1} {A}_{i} )}^{T} - \frac{1}{2} {γ}_{{γ}_{i}}^{kT} ] {\hat{Φ}}^{+} )}^{T} \qquad ( 22 )$$

and \hat{Φ}⁺ represents the pseudo-inverse.
Proof Dropping indices of (19c), becomes

$${p}^{*} = {arg min}_{p} quad ( p ) + λ {‖ p ‖}_{1} \\ \begin{matrix}quad ( p ) {=}^{Δ} {p}^{T} ( {Φ}^{T} Φ + \frac{1}{2} ρ I ) p + ( {γ}^{T} - 2 {y}^{T} Φ - {ρ ( BA )}^{T} ) p \\ = {p}^{T} {\hat{Φ}}^{T} \hat{Φ} p + ( {γ}^{T} - 2 {y}^{T} Φ - {ρ ( BA )}^{T} ) p\end{matrix} \qquad ( 23 )$$

where (23) follows from performing a Cholesky decomposition to build a unique \tilde{Φ} ∈ [symbol] ^{d×d} and then zero padding to build \hat{Φ} ∈ [symbol] ^{n×d} the relationship given in (22). Then the square is completed in order to get an equation of the form

∥\hat{Φ}p∥₂²−2ŷ^{T}\hat{Φ}p+∥ŷ∥₂²=∥ŷ−\hat{Φ}p∥₂²:

−2_{ŷ}^{T}\hat{Φ}ₚ=(γ^{T}−2_{y}^{T}Φ−ρ(BA)^{T})p.

Remark 1. The problem of finding a map S* to generate i.i.d. samples from the Bayesian LASSO posterior can be solved iteratively. Each step involves solving—in parallel—linear algebra problems and d-dimensional LASSO problems (4).

VI. An Analog-Implementable Solution to the Posterior

The results are instantiated with the Local Competitive Algorithm (LCA) first presented in which is an analog dynamical system inspired by neural image processing and exactly solves (4). This system has already been implemented in field-programmable analog arrays and integrate-and-fire neurons, thus showing promising results for reduced energy in hardware implementations. Any solver for (4) is compatible with the exemplary framework; here, the existence of a compatible solver is demonstrated, which shows the potential for energy-efficient computation of the full Bayesian LASSO posterior.

A. Locally Competitive Algorithm

In the LCA, a set of parallel nodes, each associated with an element of the basis Φₘ ∈ Φ, compete with each other for representation of the input. The dynamics of LCA are expressed by a set of non-linear ordinary differential equations (ODEs) which represent simple analog components. The system's steady-state is the solution to (4). Using the formulation presented in Theorem III.2, we solve (19c) by presenting the LCA dynamics in terms of ŷ and \hat{Φ}

$${\dot{u}}_{m} ( t ) = \frac{1}{τ} [ 〈 {\hat{Φ}}_{m} , \hat{y} 〉 - {u}_{m} ( t ) - {\sum }_{n \neq m} 〈 {\hat{Φ}}_{m} , {\hat{Φ}}_{n} 〉 {a}_{n} ( t ) ]$$

$${a}_{n} ( t ) {=}^{Δ} {T}_{λ} ( {u}_{n} ( t ) ) = max ( 0 , {u}_{n} ( t ) - λ )$$

T_{λ} is a thresholding function that induces local non-linear competition between nodes.

Remark 2. Here, N of these dynamical systems are run in parallel. For 1≤i≤N the ith LCA system corresponds to the ith update in (19c).

B. Spectral Analysis of Eeg Data

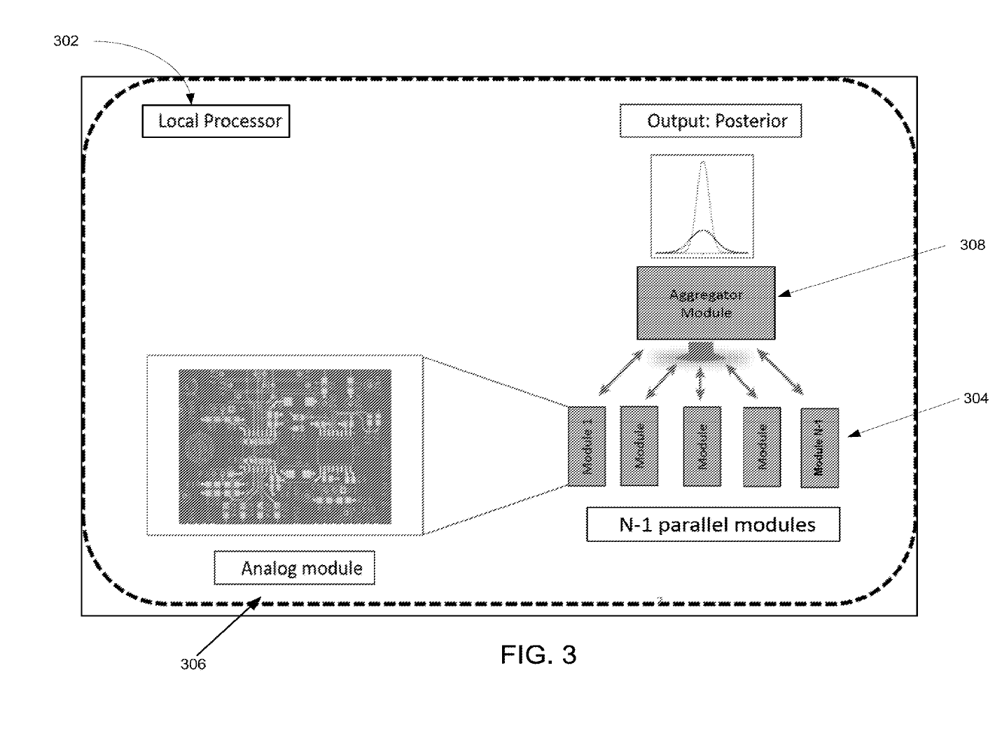

The results on the instantiation of full Bayesian LASSO simulating N parallel LCA systems is presented. Two cases of spectral analysis of electroencephalogram (EEG) recordings are shown using the exemplary framework. In both instances, in (1b), Y is a time series of band-limited EEG; Φ contains the sin and cos functions of the Fourier representation of a signal, and X represents the coefficients. The sparsity assumption on X follows since for these EEG applications, most power is concentrated in a small number of frequency bands. Furthermore, recent work has shown that a sparse assumption on an EEG spectrum can lead to higher time and frequency resolution than standard spectral analysis methods. In the first instance, EEG is recorded using epidermal electronics and performed two separate Bayesian inference problems: one under the condition of eyes open (no alpha waves) and another under eyes closed (alpha waves). As shown in FIG. 4, the Fourier coefficients sampled from the posterior under eyes closed (alpha waves) are larger in amplitude as compared to when eyes open (no alpha waves).

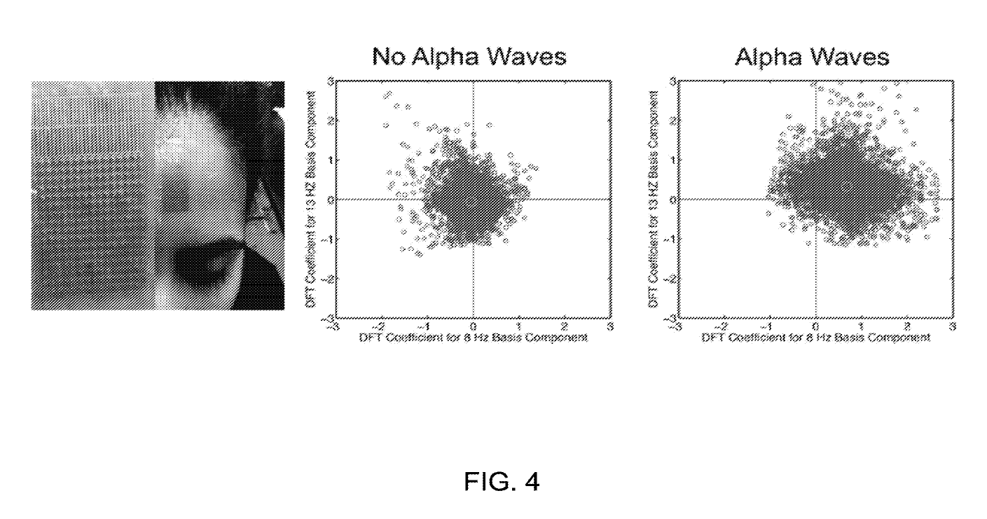

In the second instance, sleep EEG recordings from PhysioNet.com was analyzed and seven equally-spaced frequency bands from 0.1 Hz-50 Hz were considered. Two 30-second windows corresponding to light and REM sleep respectively were picked. For both windows, samples were generated from the posterior within the 95% Bayesian credibility intervals, which can be obtained from the optimal map. FIGS. 5A and 5B shows that the two groups of samples visually separate (left) and that the empirical losses for optimal decision making, when using the log loss (whose use can be justified from Bayesian decision theory), are more concentrated towards zero when incorporating the full posterior—as compared to the Bayes optimal point estimate (right).

VII. Applications

The exemplary embodiments have been implemented within a portable, multi-core processor (e.g. the Parallela system). The exemplary embodiments are also at the state of development in a Graphics Unit Processing (GPU) solution, which also allows for parallelized computation. These can be implemented in wearables, mobile phones, or tablets.

In addition, the exemplary embodiment has been simulated at the state of a working computer model simulation of an analog solver. The analog solver is comprised of N circuits that solve point estimation problems. This suggests it can be implemented in future analog hardware systems that are being established in industry.

The commercial applications of this product encompass a wide variety of mobile applications. Many current applications where a decision has to be made based on data collected within a mobile device that rely on communication with external servers can be replaced by the exemplary framework.

The exemplary embodiments can be used in medical mobile applications for health monitoring and patient compliance. A sensor takes physiological data from a patient and the patient can monitor their own condition, the device can send a summary of the state to the doctor via wireless transmission for further analysis.

VIII. Finding the Bayesian Lasso Parameter

The parameter of the standard LASSO in (5), λ, can be chosen by cross-validation, generalized cross-validation, and ideas based on unbiased risk minimization. A simplified Expectation Maximization algorithm is proposed to calculate a marginal Maximum Likelihood Estimate of λ. Moreover, the exemplary methodology allows for drawing of uncorrelated samples from the posterior leading to faster convergence of a Monte Carlo EM algorithm as compared to the Bayesian LASSO Gibbs sampling.

In the usual Expected Maximization framework, A has a likelihood function that may be maximized to obtain an empirical Bayes estimate given by

p(y|λ)=∫ₓp(x,y|λ)dx (24)

The E-step of the kth iteration involves taking the expected values (with respect to the posterior distribution) of the data log likelihood under the iterate λ^{k} to get

Q(λ|λ^{k})=log(−λ)+E_{λk}[log(p(x|λ)]+C (25)

where C represents terms not involving λ.

Thus the sequence

$${λ}^{k + 1} = {arg max}_{λ} Q ( λ \vert {λ}^{k} ) \qquad ( 26 )$$

converges to the MLE of λ. Automatic because the expectation is taken with respect to the posterior. Thus we need only sample z₁, . . . z_{N} from the posterior

For the exemplary Bayesian Lasso the steps are as follows:

1) Choose an initial λ⁽⁰⁾

2) Generate N samples from the posterior distribution as zⱼ=S(xⱼ) for j=1 . . . N where xⱼ is a sample from the prior in (3).

3) (E-Step):Approximate the expected complete data log likelihood by substituting averages for the expectation in

4) (M-step) Let λ^{k+1} be the value of λ that maximises the expected log likelihood of the previous step.

5) Return to step 2 until convergence.

The M-step leads to a simple analytical solution.

$${λ}^{k + 1} = \frac{1}{{\sum }_{i = 1}^{d} {E}_{{λ}^{k}} [ \vert {x}_{i} \vert ]} \qquad ( 27 )$$

Where the expectation is calculated with samples from the posterior distribution.

The proposed EM algorithm was ran using samples from the Bayesian LASSO Gibbs sampler (ran for 1000, 10,000, 100,000 iterations) by Park and Casella, and with the exemplary independent samples from the posterior distribution of the proposed Bayesian Lasso. The EM algorithm converges faster (for a certain degree of convergence)for samples from the exemplary proposed Bayesian Lasso as the samples are truly uncorrelated.

IX. Comparison to other Approaches

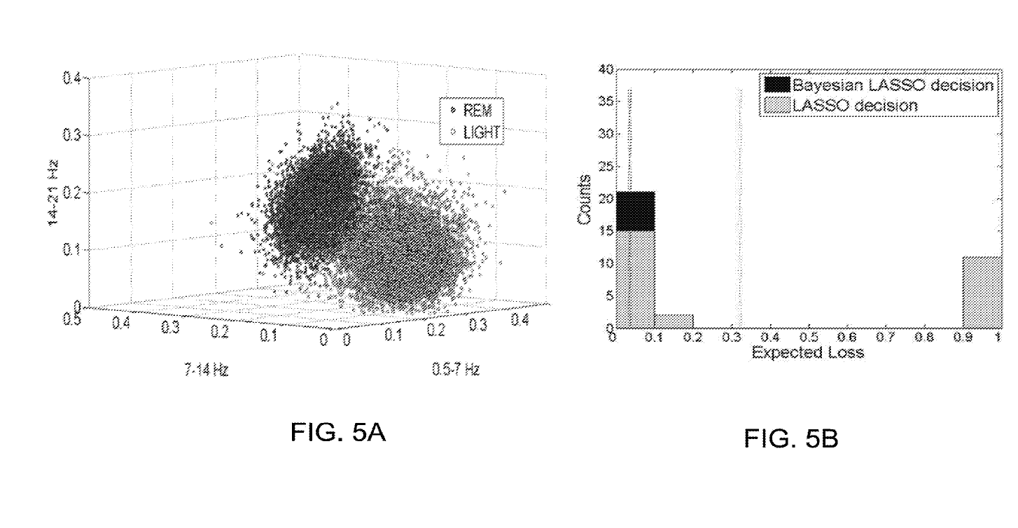

FIG. 6 compares the exemplary Bayesian Lasso posterior median estimates with the ordinary Lasso and the Gibbs sampler posterior median estimates. The vector of posterior medians is taken so that that it minimizes the L₁ norm loss average over the posterior. For both the exemplary proposed method and the Gibbs sampler method, the Bayesian Lasso estimates were computed by sweeping over a grid of λ values. The Bayesian Lasso was run with a PCE order of 3 , and N=500. The specifications for the Gibbs sampler were to use a scale-invariant prior on ρ² and run for 1000 iterations of burn-in.

Despite the Bayesian-frequentist approach, the L₁ paths are very similar to the Bayesian Lasso with a prior on ρ². The Bayesian Lasso paths are smoother than the Lasso estimates.

X. Efficient Implementation and Applications

The fundamentally parallel nature of the exemplary Bayesian Lasso formulation allows for solution implementation on a variety of platforms. A case-study is presented for platforms that can be used in applications for wearable electronics and the internet-of-things. This patent document suggests a framework for these applications to bypass large-scale transmission of data and transmit a concise and complete representation of the data (the posterior distribution) only at infrequent events.

In order to leverage the parallel nature of the algorithm presented above, in an exemplary embodiment, this patent document present implementation with a Graphics Processing Unit solution as well as an analog implementable solution. These implementations attest to the capability of Bayesian learning within currently relevant applications such as wearable electronics and the internet-of-things.

A. GPU Implementation

In the last several years Graphical Processing Units (GPUs) have gained significant attention for their parallel programmability. Multiple libraries and frameworks have evolved to assist in solving parallel problems. One such library is ArrayFire (REF) which provides a user friendly framework for building highly parallel applications. Beyond abstracting the low level programming tasks which can be cumbersome for GPUs, ArrayFire also provides highly parrallelized and optimized linear algebra algorithms.

Using ArrayFire, a generalized iterative re-weighted least-squares (GIRLS) algorithm was implemented to solve N LASSO problems of dimension d. The GIRLS algorithm requires solving only least-squares sub-problems with linear algebra operations.

FIG. 4 shows the results of electroencephalography (EEG) signal analysis using the exemplary GPU solver. In typical EEG signals, the majority of the power is concentrated in a small number of frequency bands. The EEG was recorded using epidermal electronics and two separate Bayesian inference problems were performed: one under the condition of eyes open (no alpha waves) and another under eyes closed (alpha waves). Y represents the time series from band-limited EEG, and X are the Fourier coefficients of the data. Due to the concentration of power in EEG, the sparsity assumption in X is preserved.

XI. Conclusion

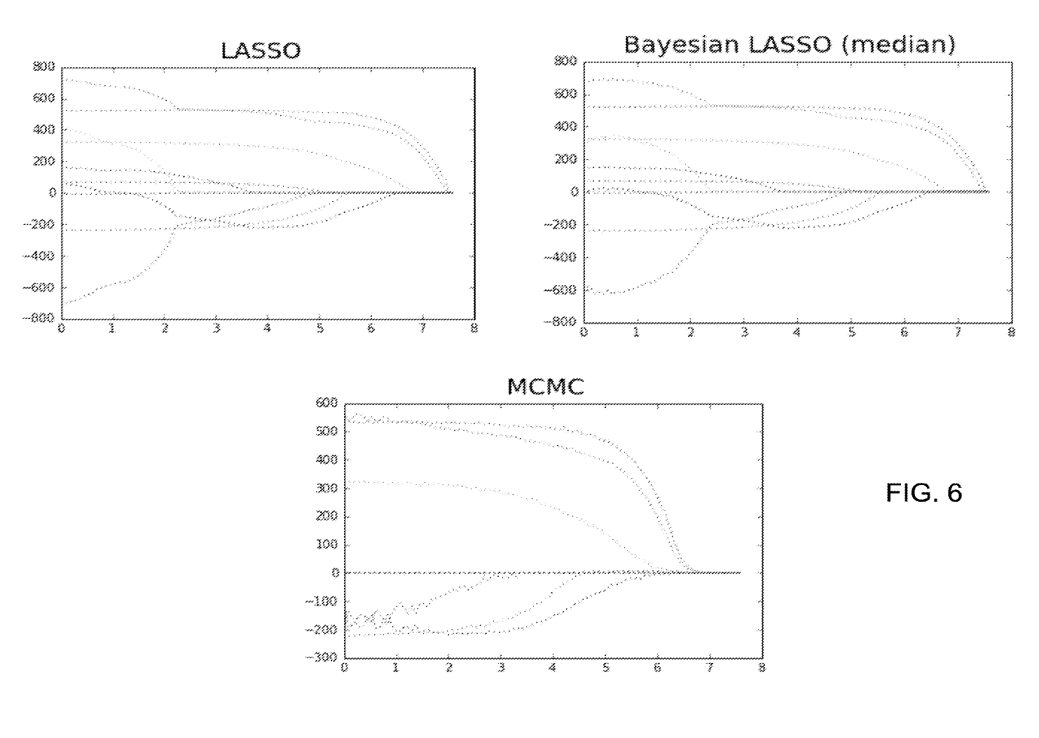

FIG. 7 illustrates an exemplary block diagram of the various components of exemplary device. In an exemplary embodiment, a mobile device (700) can determine uncertainty quantification of physiological data. The mobile device (700) includes one or more sensors (702) that can gather biometric data, a processing unit (704) connected to the one or more sensors (702) and capable of executing an uncertainty quantification algorithm on the gathered biometric data, a wireless transceiver (706) connected to the processing unit, and a display (708) connected to the processing unit (704).

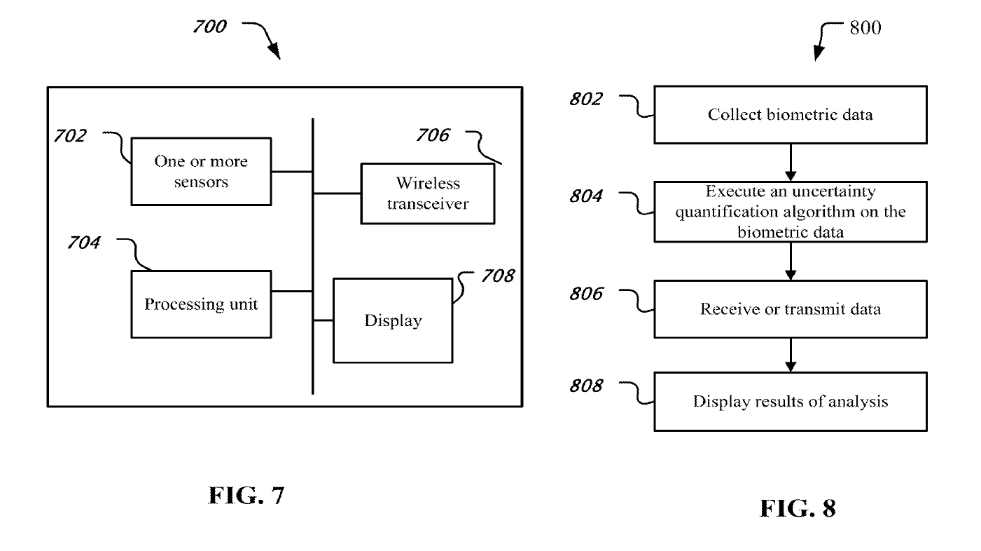

FIG. 8 illustrates an exemplary flow diagram of a method 800 implemented by an exemplary device. In an exemplary embodiment, biometric data can be collected in step 802. In step 804, an uncertainty quantification algorithm can be executed on the biometric data. In step 806, data related to the processed data can be transmitted or received. In step 808, the results of the analysis can be presented on a display.

A distributed and scalable algorithm is presented that finds the Bayesian LASSO posterior by finding a map which transforms samples from the prior into samples from the posterior. The exemplary approach only requires iteratively implementing linear algebra updates and LASSO point estimation updates. The exemplary framework was instantiated within an ‘analog-to-information’ context by finding the optimal map with a low-energy, analog-implementable LASSO solver. Consistent with FIG. 1C, this suggests that the optimal map can be found within an energy-constrained device, and only the coefficients pertaining to the map need be wirelessly transmitted. Moreover, this can facilitate optimal decision-making for any Bayesian decision-making problem: performing empirical risk minimization using posterior samples as in (5) can be done in the cloud. Future work can entail developing more energy-efficient architectures, and/or developing algorithms that only transmit when the posterior distribution reflects an abnormality.

The Bayesian Lasso can be solved with a measure transport framework by finding a map that translates samples from the Laplacian prior to the posterior distribution. A map can be produced that once computed can always be used to generate more samples from the posterior distribution simply by drawing samples from the Laplacian prior distribution. There is a clear advantage to drawing i.i.d. samples from the Bayesian Lasso posterior that is seen in the faster convergence of the EM algorithm to select the lasso parameter. Similarly, other hierarchical methods that are dependent on marginalization and computation of moments can experience a boost in convergence.

A parallelized version of the exemplary algorithm is shown that requires only Lasso solvers and linear algebra libraries for implementation. This formulation enables the leverage of the diversity of lasso solvers to compute the full Bayesian Lasso posterior.

The exemplary embodiments show the first steps of implementation of a GPU and an energy efficient architecture. Further iterations of implementation can lead to energy efficient and fast algorithms that house Bayesian LASSO in embedded systems for use in medical applications/wearable electronics.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A mobile device for determining uncertainty quantification of biometric data, the mobile device comprising:
one or more sensors capable of collecting biometric data;
a processing unit electrically coupled to the one or more sensors and capable of executing an uncertainty quantification algorithm on the biometric data collected by the one or more sensors, wherein the uncertainty quantification algorithm is capable of finding a posterior distribution or determining a quantification of uncertainty;
a wireless transceiver electrically coupled to the processing unit; and
a display operatively connected to the processing unit.

2. (canceled)

3. The mobile device of claim 1, wherein the wireless transceiver is capable of wirelessly sending a representation of the posterior distribution to a cloud server.

4. The mobile device of claim 1, further comprising:
an actuator capable of one or more of receiving a representation of the posterior distribution, performing an action, or outputting a signal received by the mobile device, wherein the actuator capable of performing the action comprises calculating an optimal action based upon the posterior distribution, and wherein the actuator includes any one or more of a speaker, a visual display, a drug delivery mechanism, and an electrical stimulator.

5. (canceled)

6. (canceled)

7. The mobile device of claim 1, wherein the display is enabled to represent the posterior distribution.

8. The mobile device of claim 1, wherein the uncertainty quantification algorithm includes a Bayesian inference algorithm.

9. The mobile device of claim 1, wherein the one or more sensors comprise electrocardiograph (EKG) monitors, adhesive-integrated flexible electronics for recording physiologic signals, or electroencephalograph (EEG) epidermal electronics.

10. (canceled)

11. (canceled)

12. The mobile device of claim 1, wherein the one or more sensors are enabled to measure one or more of maternal temperature, fetal heart rate, fetal movement, or uterine contractions.

13. (canceled)

14. The mobile device of claim 1, wherein one or more of the display or the sensors are enabled to generate an alert based on the quantification of uncertainty.

15. (canceled)

16. The mobile device of claim 14, wherein
the one or more of the display or the sensors is enabled to display a green light when the quantification of uncertainty is within a range;
the one or more of the display or the sensors is enabled to display a yellow light when the quantification of uncertainty is close to a threshold of a range; and
the one or more of the display or the sensors is enabled to display a red light when the quantification of uncertainty is outside a range.

17. (canceled)

18. (canceled)

19. The mobile device of claim 1, wherein the processing unit is enabled to receive and process one or more of tolerance settings or a range of quantification of uncertainty from a remote device.

20. (canceled)

21. (canceled)

22. (canceled)

23. (canceled)

24. (canceled)

25. (canceled)

26. The mobile device of claim 1, wherein the biometric data includes physiologic time series data.

27. (canceled)

28. (canceled)

29. (canceled)

30. A method of determining uncertainty quantification of biometric data implemented by a mobile device, comprising:
receiving biometric data from one or more sensors;
executing an uncertainty quantification algorithm on the received biometric data,. wherein the uncertainty quantification algorithm is capable of finding a posterior distribution or determining a quantification of uncertainty;
displaying the posterior distribution or generating an alert based on the quantification of uncertainty.

31. The method of claim 30, wherein the uncertainty quantification algorithm includes a Bayesian inference algorithm.

32. The method of claim 30, wherein the one or more sensors comprise electrocardiograph (EKG) monitors, adhesive-integrated flexible electronics for recording physiologic signals, or electroencephalograph (EEG) epidermal electronics.

33. The method of claim 30, wherein the one or more sensors are enabled to measure one or more of maternal temperature, fetal heart rate, fetal movement, or uterine contractions.

34. The method of claim 30, wherein the biometric data includes physiologic time series data.

35. A computer-readable program storage medium having code stored thereupon, the code, when executed by a processor, causing the processor to implement a method comprising:
receiving biometric data from one or more sensors;
executing an uncertainty quantification algorithm on the received biometric data, wherein the uncertainty quantification algorithm is capable of finding a posterior distribution or determining a quantification of uncertainty;
displaying the posterior distribution or generating an alert based on the quantification of uncertainty.

36. The computer-readable program of claim 35, wherein the uncertainty quantification algorithm includes a Bayesian inference algorithm.

37. The computer-readable program of claim 35, wherein the one or more sensors comprise electrocardiograph (EKG) monitors, adhesive-integrated flexible electronics for recording physiologic signals, or electroencephalograph (EEG) epidermal electronics.

38. The computer-readable program of claim 35, wherein the one or more sensors are enabled to measure one or more of maternal temperature, fetal heart rate, fetal movement, or uterine contractions.

39. The computer-readable program of claim 35, wherein the biometric data includes physiologic time series data.
